(19) United States
(12) Patent Application Publication
(10) Pub. No.: US 2014/0356293 A1
(43) Pub. Date: Dec. 4, 2014

(54) FLUORESCENT COMPOSITIONS WITH ENHANCED FLUORESCENCE AND METHODS BASED THEREON

(71) Applicant: The Research Foundation of the State University o New York, Albany, NY (US)

(72) Inventors: Jay E. Reeder, Syracuse, NY (US); Jed-Sian Cheng, Syracuse, NY (US); Yachao Zhang, Syracuse, NY (US); Ronald W. Wood, Rochester, NY (US)

(73) Assignee: The Research Foundation of the State of University of New York, Albany, NY (US)

(21) Appl. No.: 14/373,166

(22) PCT Filed: Jan. 18, 2013
(86) PCT No.: PCT/US2013/022258

§ 371 Date: Jul. 18, 2014

Related U.S. Application Data
(60) Provisional application No. 61/589,122, filed on Jan. 20, 2012.

(51) Int. Cl. A61K 49/00; A61K 47/46

(52) U.S. Cl. CPC A61K 49/0034; A61K 49/0091; A61K 47/46; C09K 2211/1096
USPC 424/9.6

(57) ABSTRACT

Fluorescent compositions with enhanced fluorescent intensity are provided. Methods for using the fluorescent compositions in medical imaging are also provided. The enhanced fluorescent compositions can be a mixture or solution comprising a fluorescent dye and a light-scattering emulsion or fluorescence-enhancing diluent. The fluorescent dye, e.g., indocyanine green (ICG), is activated by near-infrared radiation. The fluorescence-enhancing diluent can be milk, infant formula, intravenous fat emulsions, soy bean oil, egg phospholipids, Intralipid, Liposyn, Nutralipid, Soyacal, Travamulsion, SMOFlipid, Clinoleic, Lipovenoes and/or combinations thereof. The fluorescent intensity of the enhanced fluorescent composition can be 5 to 20 or more times greater than that of the fluorescent dye in solution without added fluorescence-enhancing diluent.

### CROSS-REFERENCE TO RELATED APPLICATIONS

This application claims priority to and the benefit of co-pending U.S. provisional patent application Ser. No. 61/589,122, entitled “Enhancement of near infrared fluorescence using milk for visualization of ureteral anatomy during pelvic surgeries,” filed Jan. 20, 2012, which is incorporated herein by reference in its entirety.

### STATEMENT REGARDING FEDERALLY SPONSORED RESEARCH OR DEVELOPMENT

The disclosed invention was made in part with government support under contract no. 5R01DK75036-4 from the National Institutes of Health. The government has rights in this invention.

### 1. TECHNICAL FIELD

The present invention relates to fluorescent compositions for visualizing anatomical structures such as tissues or organs. The invention further relates to methods for using fluorescent compositions for visualizing anatomical structures such as tissues or organs. The invention further relates to imaging methods using fluorescent compositions. The invention further relates to imaging methods during nephrostomy, hysterectomy, bariatric, cancer and other surgeries.

### 2. BACKGROUND OF THE INVENTION

Recent technological advances in illumination devices and digital image acquisition have led to rapid growth in the use of fluorescence in medical applications (Rao 2007). Near-infrared fluorescent (NIRF) agents are particularly attractive for in vivo use due to the fact that near-infrared (NIR) light penetrates biological tissues better then the visible wavelengths and can be imaged at depth (Duck 1990). The Near-InfraRed Fluorescent (NIRF) agents have been quickly adapted for in vivo fluorescence imaging studies using a sensitive camera. Indocyanine green (ICG) is an FDA-approved fluorescent carbocyanine dye used in medicine as an indicator substance and in NIRF imaging. Indocyanine green is also known as Cardiogreen, Foxgreen, Cardio-Green, Fox Green, IC Green. It is normally supplied as a powder and is reconstituted in sterile water prior to intravenous injection and is labeled for use within six hours after reconstitution. ICG sodium salt is normally available in powder form and can be dissolved in various solvents; 5% (<5% depending on batch) sodium iodide is commonly added to ensure better solubility (Augustin et al. 2001). The sterile lyophilisate of a water-ICG solution is approved many European countries and the United States under the name ICG-Pulsion® (manufacturer: Pulsion) and IC-Green® (manufacturer: Akorn) as a diagnostic for intravenous use.

The absorption and fluorescence spectrum of ICG is in the near infrared region. The quantum efficiency, and excitation and emission spectra of ICG depend largely on the solvent used and the concentration. ICG absorbs mainly between 600 nm and 900 nm and emits fluorescence between 750 nm and 950 nm. The large overlap of the excitation and emission spectra can lead to marked reabsorption of the emission by ICG itself. Maximum fluorescence wavelengths are approximately 810 nm in water and approximately 830 nm in blood. The maximum absorption is ˜800 nm in plasma at low concentrations. In combination with fluorescence detection, lasers or filtered light sources with a wavelength of 760-806 nm are typical. At this wavelength, ICG absorbs well and selective imaging at longer wavelengths is possible.

ICG is used in medical diagnostics, including for determining cardiac output, hepatic function, and liver blood flow, and is also used extensively for fluorescence ophthalmic angiography. ICG has a long history of use as a test of cardiac output and liver function as it is excreted exclusively in the bile and has a serum half-life of 3 to 4 minutes after intravenous administration. ICG use allows non-invasive monitoring of liver or splanchnic perfusion (by monitoring the changes in the ICG plasma disappearance rate, this method is suitable as a parameter for predicting the probability of survival of intensive-care surgical patients). Several new surgical applications relying on NIRF angiography have been reported, including: plastic surgery—skin and muscle transplants, and determination of amputation level; abdominal surgery—gastrointestinal anastomosis; general surgery—wound healing and ulcers; internal medicine—diabetic extremities; heart surgery—aortocoronary bypasses; neurology—a tracer in cerebral perfusion diagnostics; and with stroke patients—monitoring. NIRF is also used in the context of sentinel lymph node identification and harvest, replacing radionuclides and blue dye in surgery for breast cancer, malignant melanoma and gastrointestinal tumors. NIRF imaging eliminates the problems of obtaining, application and disposing of radionuclides and the 1% risk of anaphylaxis from blue dye (Hirche, 2010a, 2010b). NIRF has been used in the diagnosis and management of rheumatic diseases.

A number of imaging systems have been developed by industry. Novadaq Technologies introduced the SPY system to determine intraoperatively the patency of cardiac bypass grafts. The system images without ionizing radiation and reduces the need for a second operative intervention. NIRF imaging capabilities have been added to the DaVinci robotic surgery system, and Novadaq, Storz, and others have laparoscopic systems in development or in clinical use. NIRF imaging has also been incorporated into the Zeiss Pentero operating room microscope for video angiography.

ICG used to image the urinary tract of rats was prepared according to package insert instructions (aqueous solution 2.5 mg/ml) and diluted to 10 μg/ml to achieve peak fluorescence. It was observed that the fluorescence of these preparations was not as intense as that achieved when ICG was injected intravenously and diluted into the blood.

Milk has a long history of use in surgery to detect leaks from pelvic lumens. The method of instilling milk (sterile infant formula Similac) in the urinary tract to document watertight urothelial closure was reported in 1976 (McLoughlin 1976), but the practice dates back to at least 1906 (Kelly 1906) and is described in several contemporary textbooks (e.g., Kovac 2006). However, milk does not exhibit significant fluorescence in the near-IR range.

Citation or identification of any reference in Section 2, or in any other section of this application, shall not be considered an admission that such reference is available as prior art to the present invention.

### 3. SUMMARY OF THE INVENTION

An enhanced fluorescent composition is provided comprising:

a fluorescent dye; and

fluorescence-enhancing diluent (also referred to herein as “FED” or “diluent”).

In one embodiment, the enhanced fluorescent composition comprises

a fluorescent dye; and

fluorescence-enhancing diluent (“FED”) comprising a solution, emulsion or suspension fat, oil, or protein in liquid.

In another embodiment, the fluorescent composition comprises a liquid.

In another embodiment of the fluorescent composition, the liquid is water, saline solution, ethanol or DMSO.

In another embodiment of the fluorescent composition, the fluorescence-enhancing diluent is a solution of powdered, freeze-dried, dehydrated, evaporated, concentrated or condensed fat, oil, or casein emulsion in liquid.

In another embodiment of the fluorescent composition, the fluorescent dye is a near-infrared fluorescent dye.

In another embodiment of the fluorescent composition, the fluorescent dye is indocyanine green (ICG).

In another embodiment of the fluorescent composition, the fluorescence-enhancing diluent is milk, infant formula (e.g., Enfamil), intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

In another embodiment of the fluorescent composition, the milk is cow, sheep or goat milk.

In another embodiment of the fluorescent composition, the concentration of fluorescence-enhancing diluent in the fluorescent composition is 0.1% to 90% v/v.

In another embodiment of the fluorescent composition, the concentration of fluorescence-enhancing diluent is between 5% and 15% (±5%) v/v

In another embodiment of the fluorescent composition, the concentration of ICG in the enhanced fluorescent composition in μg/mL is between 5% and 40% (±3%).

In another embodiment of the fluorescent composition, the ICG concentration in μg/mL is between 5% and 15% (±3%).

In another embodiment of the fluorescent composition, the fluorescent intensity of the enhanced fluorescent composition is at least 2, 3, 4, 5, 6, 7, 8, 10, 15, or 20 times that of a fluorescent solution containing the same amount of fluorescent dye but no fluorescence-enhancing diluent.

In another embodiment, the fluorescent composition is micro-encapsulated.

A method is provided for enhancing fluorescent intensity of a fluorescent dye comprising the step of mixing the fluorescent dye with fluorescence-enhancing diluent, thereby producing an enhanced fluorescent composition.

In one embodiment, the method comprises the step of mixing the fluorescent dye with a liquid.

In another embodiment of the method, the liquid is water, saline solution, ethanol or DMSO.

In another embodiment of the method, the liquid is water, saline solution, ethanol or DMSO.

In another embodiment of the method, the fluorescent dye is a near-infrared fluorescent dye.

In another embodiment of the method, the fluorescent dye is indocyanine green (ICG).

In another embodiment of the method, the fluorescence-enhancing diluent is milk, infant formula (e.g., Enfamil), intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

In another embodiment of the method, the milk is cow, sheep or goat milk.

In another embodiment of the method, the fluorescence-enhancing diluent is a solution of powdered, freeze-dried, dehydrated, evaporated, concentrated or condensed fat emulsion in liquid.

In another embodiment of the method, the concentration of fluorescence-enhancing diluent in the fluorescent composition is 0.1% to 90% v/v.

In another embodiment of the method, the concentration of fluorescence-enhancing diluent is between 5% and 15% (±5%) v/v

In another embodiment of the method, the fluorescent dye is ICG and the concentration of ICG in μg/mL is between 5% and 40% (±3%).

In another embodiment of the method, the ICG concentration in μg/mL is between 5% and 15% (±3%).

In another embodiment of the method, the fluorescent intensity of the enhanced fluorescent composition is at least 2, 3, 4, 5, 6, 7, 8, 10, 15, or 20 times that of a fluorescent solution containing the same amount of fluorescent dye but no fluorescence-enhancing diluent.

In another embodiment, the method comprises the step of micro-encapsulating the enhanced fluorescent composition.

A method is also provided for performing a nephrostomy on an animal or human patient or subject comprising the steps of:

exposing a kidney of the patient or subject surgically in the retroperitoneum;

exposing the renal pelvis and the proximal ureter of the kidney surgically;

inserting a tube into the midpole of the kidney;

introducing (e.g., injecting) an enhanced fluorescent composition into the tube;

imaging the tube and the kidney;

evaluating or measuring the position of the tube or the connection of the tube with respect to the kidney;

if the connection of the tube to the kidney is judged to be poor, repositioning the tube and repeating the previous steps;

fixing the tube in place (using methods known in the art, e.g., such as with a cyanoacrylate glue); and

replacing or repositioning the kidney in the retroperitoneum and surgically closing the animal.

A method is also provided for visualizing lumens (e.g., bladder or ureter), the method comprising the steps of applying an enhanced fluorescent composition to the lumen; and imaging lumen to which the enhanced fluorescent composition is applied.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated, enlarged, exploded, or incomplete to facilitate an understanding of the invention.

FIG. 1. Fluorescence of ICG in reconstituted dry milk. See Example 1 for details.

FIG. 2. White light image of ureteral catheter inserted into ureter of cadaver. See Example 1 for details.

FIG. 3. NIRF image of ureteral catheters inserted into the ureters of a cadaver. One catheter is filled with ICG-milk enhanced fluorescent composition. See Example 1 for details.

FIG. 4. Time lapse of retroperitoneal filling of renal pelvis with ICG-milk visualized using NIRF in human cadaver. See Example 1 for details.

FIG. 5. Relative fluorescence intensity of ICG in Enfamil prepared from 2.5 mg/ml IC Green in DMSO stock. Peak fluorescence was observed at 7.8 μg/ml and a final DMSO concentration of 0.3%. See Example 1 for details.

FIG. 6. Demonstration of kidney injury in live pig using ICG-Enfamil. The ureter and renal pelvis were filled by retrograde injection of ICG-Enfamil into a ureteral catheter. The renal pelvis is clearly identified in the time-lapse NIRF images. A scalpel was used to make a small incision in the kidney resulting in leakage and pooling of ICG-Enfamil. See Example 1 for details.

FIG. 7. Intraperitoneal injection and bladder instillation of ICG-Intralipid. A. Reflected 830 nm light. B. ICG-Intralipid instilled in the bladder by catheter. Bladder is visible in addition to the persistent abdominal fluorescence. C. ICG-Intralipid injected intraperitoneally (IP). D. Midline incision confirms the pooling of the ICG-Intralipid in the abdomen and retention in the bladder. See Example 1 for details.

FIG. 8. Biodistribution of intraperitoneal ICG-Intralipid. The top left image is reflected near infrared light showing the position of the mouse in the imaging system. The top middle image was captured after injection of ICG-Intralipid which outlines loops of intestine, stomach and the bladder. At top right the gall bladder can be seen as an intense spherical object attached to the liver at necropsy. The bottom panels are reflected light, NIRF imaging of bowel contents and finally a gray-scale version of a merged pseudocolor image of the first two images. See Example 1 for details.

FIG. 9. Results of imaging of ICG solutions. See Example 2 for more details.

FIG. 10. Images captured as described for FIG. 9, with the inclusion of a floating toothpick to provide an object with sharp edges to obtain optimal focus. ICG was dissolved in DMSO and then diluted into Enfamil or Intralipid. Concentrations decrease from right to left. The intensity of ICG fluorescence in Enfamil is not saturated and the peak around 8 μg/ml can be seen. Under these conditions, fluorescence saturated in Intralipid, but a peak at 8 μg/ml can be seen by reducing the exposure time from 90 to 70 ms. See Example 2 for more details.

FIG. 11. Graphical presentation of the fluorescence values (modes) in the image montages from FIG. 10. See Example 2 for more details.

FIG. 12. Stability of fluorescence in enhanced fluorescent composition. See Example 2 for more details.

### 5. DETAILED DESCRIPTION OF THE INVENTION

### 5.1 Fluorescent Composition with Enhanced Fluorescent Intensity

Fluorescent compositions with enhanced fluorescent intensity (also referred to herein as “enhanced fluorescent composition,” “enhanced fluorescent solution,” or “enhanced fluorescent dye solution”) are provided. Diluents (also referred to as fluorescence-enhancing diluents or “FED”) for use in the preparation of enhanced fluorescent dye solutions are also provided. Enhanced fluorescent compositions for use in medical imaging and procedures, and methods for using such enhanced fluorescent compositions in medical procedures are also provided.

The fluorescent composition with enhanced fluorescent intensity can be a mixture or solution comprising a fluorescent dye and a fluorescence-enhancing diluent. Some embodiments of these formulations increase quantum efficiency of the fluorescent dye (e.g., ICG) using only medically safe constituents. In one embodiment, the fluorescence-enhancing diluent is Intralipid, which is an emulsion. In one embodiment, the fluorescence-enhancing diluent is non-fat milk, which is a suspension.

In one embodiment, the fluorescence-enhancing diluent is a fat other than milk and is a colloidal emulsion. An emulsion is a dispersion of a liquid in a liquid, not to be confused with dissolving a liquid into a liquid (such as alcohol in water).

In one embodiment, the fluorescent composition can be made, using standard techniques, by mixing liquid, powdered, dehydrated, freeze-dried, evaporated, or concentrated milk (non-fat, low-fat, normal fat) with a saline solution or with water to produce a diluent. This diluent is then mixed with a solution of fluorescent dye.

In one embodiment, the fluorescent dye can be indocyanine green (ICG), or other carbocyanine dye and/or combinations thereof.

In another embodiment, the fluorescent dye is activated by near-infrared radiation. Near-infrared fluorescent (NIRF) dyes, which are well known in the art, are particularly preferred. The wavelengths that activate fluorescent dyes are commonly known in the art.

In one embodiment, the diluent is a fat emulsion, such as a solution of powdered, freeze-dried, dehydrated, evaporated, concentrated or condensed fat, oil, or casein emulsion in liquid.

In another embodiment, the fat emulsion is milk, infant formula (e.g., Enfamil), intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

Medically approved fat emulsions can be safely used as a solvent or diluent for enhancing the fluorescence intensity of a fluorescent dye. The fluorescent dye can be mixed with the fat emulsion and stored in pre-mixed form, or can be mixed from stock solutions just before its use.

In another embodiment, the fat emulsion solution can be powdered, freeze-dried, dehydrated, evaporated, concentrated or condensed fat or fat emulsion mixed with a liquid such as water, saline solution, ethanol, or dimethyl sulfoxide (DMSO). Other suitable liquids can be used that are known in the art for dissolving fat and/or fluorescent dyes for medical purposes.

In a specific embodiment, the diluent is mammalian milk, such as bovine (cow) milk, goat milk, or sheep milk, can be employed. Milk can be safely used as a solvent or diluent for enhancing the fluorescence intensity of a fluorescent dye. Certain milks, such as bovine milk, are used for procedures such as for leak detection after surgery. The milk can be a solution prepared from powdered, freeze-dried, dehydrated, evaporated, or concentrated milk mixed with a liquid such as water or a saline solution.

In another embodiment, the concentration of diluent, such as fat emulsion or milk, in the fluorescent composition can range from 0.1% to 90% v/v. In specific embodiments, the concentration of fat emulsion in 1%, 2%, 3%, 4% or 5% (±0.5%) v/v.

In another embodiment, the concentration of diluent, such as fat emulsion or milk, in the fluorescent composition is can range from 5-10% (±5%) v/v.

In another embodiment, the concentration of diluent, such as fat emulsion or milk, in the fluorescent composition is can range from 10-95% (±5%) v/v.

In a specific embodiment, Intralipid solutions can be used with small but easily measured amounts of ICG in solution. For example 1 cc of a ICG/Ethanol solution can be added to 100 ml of Intralipid.

In a specific embodiment, the concentration of milk in the fluorescent composition is between 5% and 15% (±3%) or between 5% and 15% (±3%).

In another embodiment, the concentration of ICG in the enhanced fluorescent composition in μg/mL is between 5% and 40% (±3%).

In another embodiment, the concentration of ICG is 5% (±3%) and the concentration of milk is 10% to 30%, 10% to 40%, or 10% to 50% (±5%).

In another embodiment, the ICG concentration in μg/mL is between 5% and 10% (±3%) in the enhanced fluorescent composition and the concentration of milk in the solution is between about 5% and 30% (±3%) in the enhanced fluorescent composition. In another embodiment, the ICG concentration in μg/mL is about 1, 2, 3, 4, 5, 6, 7, 8, 9, or 10% (±0.5%).

In another embodiment, the milk is added to a solution of fluorescent dye (e.g., ICG) in sufficient quantity to achieve a concentration of milk in the solution equal to 1% to 5%, 5% to 10%, 10% to 20%, 20%-30% or 30% to 40%.

In another embodiment, the ICG concentration in μg/mL is about 5% and the milk concentration is about 10%, 20%, 30%, 40%, 50%, 60%, 70% or 80%.

In another embodiment, an enhanced fluorescent composition is provided wherein the fluorescent intensity of the enhanced fluorescent composition is at least 2, 3, 4, 5, 6, 7, 8, 10, 15, or 20 times that of a fluorescent solution containing the same amount of fluorescent dye but none of the fluorescence-enhancing diluents described herein, including milk or fat emulsions.

Pre-mixed enhanced fluorescent compositions may be dried for storage using methods well known in the art.

In one embodiment, solutions of enhanced fluorescent compositions can be applied to a surface and dried to provide a fluorescent marking or coating visible using appropriate equipment and lighting conditions, such as during surgery using NIRF systems. If necessary, the layer of dried fluorescent solution can be coated with a protective layer to keep it from being degraded, changed or otherwise influenced by the environment to which the surface will be subjected.

In one embodiment, the enhanced fluorescent composition is micro-encapsulated. Micro-encapsulation can be used to maintain the solution in the liquid phase. Micro-encapsulation is a process in which tiny particles or droplets are surrounded by a coating to give small capsules many useful properties. In simple terms, a microcapsule is a small sphere with a uniform wall around it. The material inside the microcapsule is referred to as the core, internal phase, or fill, whereas the wall is sometimes called a shell, coating, or membrane. Most microcapsules have diameters between a few micrometers and a few millimeters. It is well known in the art that every class of food ingredient can be encapsulated. The technique of microencapsulation depends on the physical and chemical properties of the material to be encapsulated.

Various methods can be used to manufacture microcapsules encapsulating the enhanced fluorescent compositions provided herein, including, but not limited to, pan coating, air-suspension coating, centrifugal extrusion, using a vibrational nozzle, spray-drying, physico-chemical methods such as ionotropic gelation and coacervation, and by chemical methods such as interfacial polycondensation, interfacial cross-linking, in-situ polymerization and matrix polymerization. These techniques are well known in the art and can be readily adapted to micro-encapsulate an enhanced fluorescent composition. The types and forms of microcapsules that encapsulate enhanced fluorescent compositions, however, may bear little resemblance to simple spheres. The core of such a microcapsule may be a crystal, a jagged adsorbent particle, an emulsion, a suspension of solids, or a suspension of smaller microcapsules. The microcapsule may even have multiple walls.

Although fluorescent nano-emulsions of ICG are known in the art that are transparent and are taken up by cells (see, e.g., US 20110200532, Goutayer et al.), the enhanced fluorescent compositions are not taken up by cells, allowing them to be used to visualize luminal spaces. The emulsions and suspensions used in some embodiments of the compositions and methods disclosed herein have particles or particle aggregates that are 50 nm or greater in size, and may be as large as 0.1 micron, 0.5 micron, 1 micron, 2 microns, 5 microns, 10 microns or larger.

Further, the enhanced fluorescent compositions can behave like fluorescent dye alone in water, eventually entering the venous return, entering the liver and exiting in bile with no enterohepatic recirculation.

ICG is intensely fluorescent in blood and in some solvent solutions, but enhancement of ICG fluorescence in the systems described herein may also be a result of the light scattering properties of the particulate solution. ICG is known in the art to have no detectable access to kidney tumor tissues, and margin identification arises from fluorescence in blood contrasted against the dark tumor. Thus toxicity profiles for the ICG mixture comparable to ICG alone can be achieved, so much so that there may be no need to undergo further toxicity evaluation since the ICG mixture is essentially a trace dopant in an IV parenteral solution. Conversely, nano-emulsions of near-infrared fluorescent dyes (such as ICG) target incorporation into cells and may not be safe for use in medical applications.

In other embodiments, solvent solutions (e.g., ethanol or DMSO) remain stable for prolonged periods, and can be used instead of water or saline to formulate enhanced fluorescent compositions that have prolonged shelf stability. Intralipid has very long shelf stability under appropriate storage conditions. Thus prolonged stability may be achieved for the enhanced fluorescent compositions. Dry mixtures achieved through encapsulation or lyophilization can also be achieved readily using methods known in the art, thus permitting reconstitution before use.

### 5.2 Method of Enhancing the Fluorescence Intensity of a Fluorescent Dye

Methods are provided for enhancing fluorescent intensity of a fluorescent dye (e.g., ICG) by mixing it with a fluorescence-enhancing diluent. The enhanced fluorescent compositions comprising fluorescent dyes (e.g., ICG) and emulsions (e.g., mammalian milk, Intralipid) that are suitable for use in the methods are described hereinabove (see Section 5.1).

Fluorescence can be detected and intensity measured by photographic (silver halide) film, or electronically using photomultiplier tubes, or detected, measured, and imaged with digital cameras (CCD for example). Specificity for fluorescent emission is obtained using an interference filter that is selective for a specific wavelength range.

In certain surgical procedures, such as nephrostomy, peritoneal tissues (e.g., retroperitoneum) and/or other surrounding tissues can overly the site of surgery. The enhanced fluorescent compositions can be used for visualization during such surgical procedures, where they can be substituted for conventional fluorescent dyes and visualization agents. Despite the overlying tissues at the surgical site, the enhanced fluorescent compositions provide stronger signal-to-background ratios for visualization than do conventional fluorescent dyes without added fluorescence-enhancing diluent.

The fluorescent intensity of the enhanced fluorescent composition provided herein can be 5 to 20 or more times greater than that of the fluorescent dye in solution without added fluorescence-enhancing diluent. In certain embodiments, the signal is 5-20, 20-30, 30-40, 40-50 or 50-100 times greater than that obtained from the fluorescent dye alone in solution without added fluorescence-enhancing diluent. Fluorescence intensity can be more than 100 fold higher, perhaps significantly more than 100 fold higher, than that that obtained without the added fluorescence-enhancing diluent.

The method provided herein has many medical and surgical applications, that will be apparent to the skilled practitioner, such as: performing angiography in ophthalmology; diagnosis of corneal abrasions, corneal ulcers and herpetic corneal infection; evaluation of the tear layer under rigid gas permeable contact lenses; non-invasive monitoring of liver or splanchnic perfusion; perfusion diagnostics of tissues and organs; navigation for sentinel lymph node biopsy with tumors; angiography to diagnose and categorize vascular disorders (in, for example, legs), retinal disease macular degeneration, diabetic retinopathy, inflammatory intraocular conditions, and intraocular tumors; during surgery for brain tumors; and diagnosis of rheumatic diseases.

In one embodiment, an imaging method is provided that comprises the steps of applying an enhanced fluorescent composition to an area of interest to be imaged; and imaging the area of interest to which the enhanced fluorescent composition is applied.

In a specific embodiment, a method is provided for visualizing lumens such as the bladder and ureter comprising applying an enhanced fluorescent composition to the lumen; and imaging lumen to which the enhanced fluorescent composition is applied.

Methods are also provided for performing angiography in ophthalmology (also diagnosis of corneal abrasions, corneal ulcers and herpetic corneal infections, or to evaluate the tear layer under rigid gas permeable contact lenses); non-invasive monitoring of liver or splanchnic perfusion; perfusion diagnostics of tissues and organs; navigation for sentinel lymph node biopsy with tumors; angiography to diagnose and categorize vascular disorders (in, for example, legs), retinal disease, macular degeneration, diabetic retinopathy, inflammatory intraocular conditions, and intraocular tumors; during surgery for brain tumors; and diagnosis of rheumatic diseases. These methods comprise the steps of applying an enhanced fluorescent composition to an area of interest to be imaged; and imaging the area of interest to which the enhanced fluorescent composition is applied.

In another embodiment, the method is provided for imaging an object or system, comprising the steps applying an enhanced fluorescent composition to the object or system; and imaging the object or system to which the enhanced fluorescent composition is applied.

A method is also provided for performing a nephrostomy on an animal or human patient or subject comprising the steps of:

Retroperitoneal surgical exposure of kidney of the patient or subject

Surgical exposure of the renal pelvis and proximal ureter;

inserting a tube into the midpole (middle portion) of the kidney;

introducing (e.g., infusing) an enhanced fluorescent composition into the tube;

imaging the tube and the kidney;

determining the position of the tube or the connection of the tube with respect to the kidney;

if the connection of the tube to the kidney is judged to be poor, repositioning the tube and repeating the previous steps;

fixing the tube in place (using methods known in the art, e.g., such as with a cyanoacrylate glue); and

positioning the kidney in retroperitoneal space and surgically closing the animal.

A method is also provided for enhancing fluorescent intensity of a fluorescent dye, such as an imaging dye, comprising the step of mixing the fluorescent dye with fluorescence-enhancing diluent, thereby producing an enhanced fluorescent composition.

In one embodiment, the method comprises the step of mixing the fluorescent dye with a liquid.

In another embodiment of the method, the liquid is water, saline solution, ethanol or DMSO.

In another embodiment of the method, the liquid is water, saline solution, ethanol or DMSO.

In another embodiment of the method, the fluorescent dye is a near-infrared fluorescent dye.

In another embodiment of the method, the fluorescent dye is indocyanine green (ICG).

In another embodiment of the method, the fluorescence-enhancing diluent is milk, infant formula (e.g., Enfamil), intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

In another embodiment of the method, the milk is cow, sheep or goat milk.

In another embodiment of the method, the fluorescence-enhancing diluent is a solution of powdered, freeze-dried, dehydrated, evaporated, concentrated or condensed fat emulsion in liquid.

In another embodiment of the method, the concentration of fluorescence-enhancing diluent in the fluorescent composition is 0.1% to 90% v/v.

In another embodiment of the method, the concentration of fluorescence-enhancing diluent is between 5% and 15% (±5%) v/v

In another embodiment of the method, the fluorescent dye is ICG and the concentration of ICG in μg/mL is between 5% and 40% (±3%).

In another embodiment of the method, the ICG concentration in μg/mL is between 5% and 15% (±3%).

In another embodiment of the method, the fluorescent intensity of the enhanced fluorescent composition is at least 2, 3, 4, 5, 6, 7, 8, 10, 15, or 20 times that of a fluorescent solution containing the same amount of fluorescent dye but no fluorescence-enhancing diluent.

In another embodiment, the method comprises the step of micro-encapsulating the enhanced fluorescent composition.

The following examples are offered by way of illustration and not by way of limitation.

### 6. EXAMPLES

### 6.1 Example 1

### Enhancement of Fluorescence in Fluorescent Dye Solutions

This example describes investigations of enhanced fluorescent compositions.

Methods

Instrumentation.

Real-time near-infrared fluorescent (NIRF) imaging was accomplished using a NIRF imaging system. The ring light was coupled to a tungsten halogen source filtered with a Chroma excitation filter (775 nm). The Navitar Zoom 7000 lens was fitted with a Chroma emission filter (845 nm). Images were captured with a Qimaging Retiga camera. Petri dishes (35 mm diameter) were positioned in the imaging system that contained 2 ml of a sample of an enhanced fluorescent composition comprising ICG. This system was used in the investigations of ICG fluorescence enhancement described in this example. Excitation light was delivered to the field of interest using a fiber optic ring light coupled to a tungsten halogen source (Illumination Technologies, Inc. IT 9596-ER) filtered with a 775 nm bandpass filter (Chroma Technologies). A Navitar Zoom 7000 Lens was fitted on a Qimaging Retiga EXi cooled CCD camera.

Indocyanine Green (ICG)-Based Enhanced Fluorescent Compositions

Indocyanine Green (ICG) stock solutions were prepared by adding 10 ml of diluent to a 25 mg vial of IC Green (Akorn Incorporated). Diluents were water (supplied by the manufacturer), ethanol, or dimethyl sulfoxide (DMSO).

Milk solutions were prepared by dissolving Instant Nonfat Dry Milk (Nestle Carnation) in water at various concentrations up to 30% weight to volume

To prepare ICG-based enhanced fluorescent compositions, ICG stock solutions were added to achieve concentrations up to 250 μg/ml. Relative fluorescence intensity of various formulations was measured in 35 mm circular Petri dishes containing 2 ml. A floating toothpick provided a focusing target.

Intralipid formulations were prepared from Intralipid 20% (Fresenius Kabi), which is a sterile non-pyrogenic fat emulsion prepared for intravenous administration for parenteral nutrition. Intralipid is also a vehicle for intravenous (i.v.) delivery of propofol, and is used as an antidote for acute local anesthetic toxicity. It is a white opaque liquid containing 20% soy bean oil, egg phospholipids, glycerin, and water. ICG stock solutions were added to achieve a range of concentrations of ICG up to 250 μg/ml in the enhanced fluorescent composition.

Experimental Design and Results

Fluorescence of ICG in Reconstituted Dry Milk.

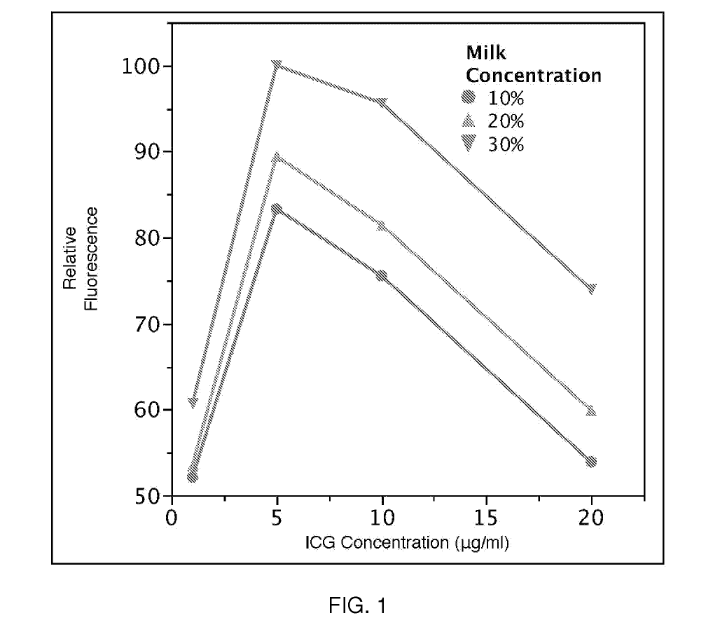

Stock aqueous ICG solution was diluted into 10, 20, and 30% milk to achieve 1, 5, 10, and 20 μg/ml final concentrations. Fluorescent images of 2 ml of each preparation in 35 mm Petri dishes were acquired using QCapture and quantified using ImageJ. Fluorescence increased with milk concentration and at all concentrations of milk, peak fluorescence was achieved at 5 μg/ml (FIG. 1); quenching occurred at concentrations of 10 μg/ml and higher. Under these conditions, milk without ICG and ICG diluted into water or saline had no detectable fluorescence.

Visualization of Ureteral Anatomy Using an ICG-in-Milk Enhanced Fluorescent Composition.

Ureteral injury is a rare but severe complication of colorectal and gynecologic surgeries due to its insidious onset and subsequent delay in detection (Brandes 2004, Alawadi 2005). Therefore, early identification and prevention becomes essential. The practice of prophylactic preoperative ureteral catheter placement is both common and controversial. The low incidence of reported ureteral injuries partially attribute to the inconclusive results in proving its benefits (Kuno 1998, Botthwell 1994, Wood 1996).

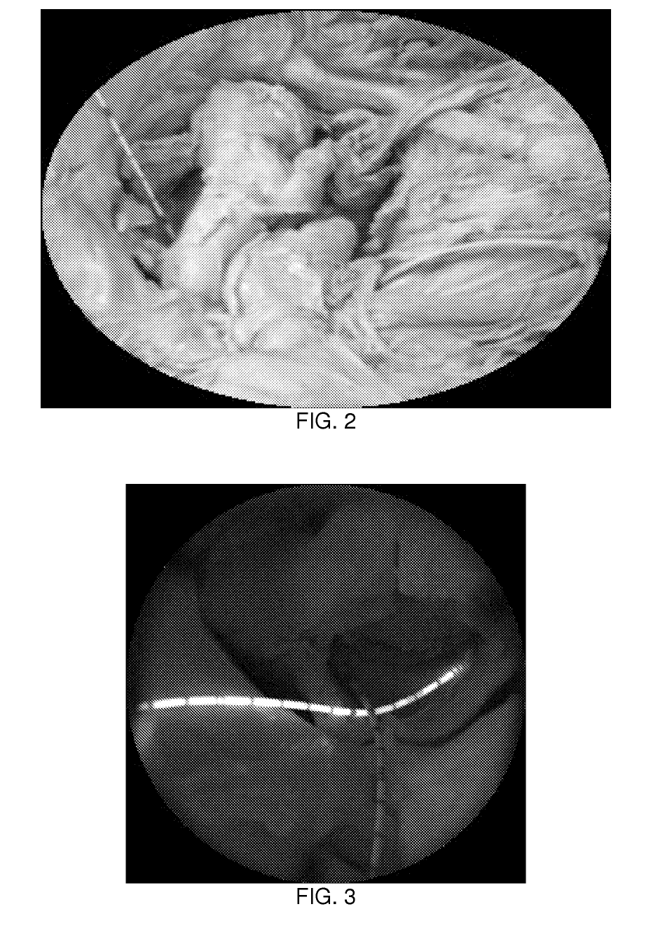

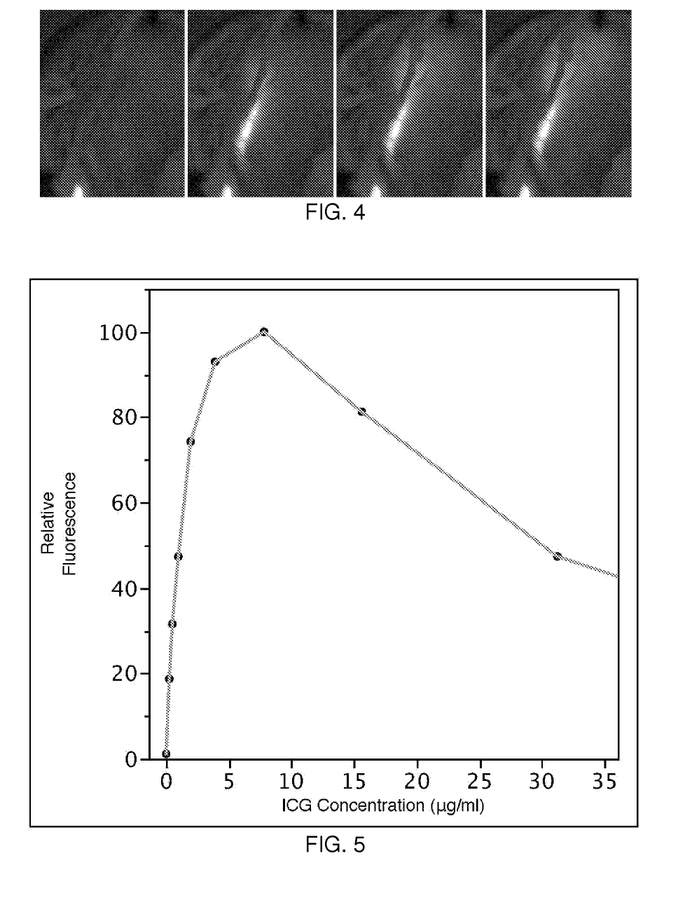

An embalmed human female cadaver, previously prepared for prosection, was provided by the Gross Anatomy Laboratory of SUNY Upstate Medical University. The bladder was opened anteriorly to allow access to the ureteral orifices (FIGS. 2 and 3). A guide wire was inserted and an open-end-flex-tip ureteral catheter was guided into the ureter over the guide wire, which was then removed. A 30 cc syringe containing an enhanced fluorescent composition consisting of 5 μg/ml ICG in 10% milk (Carnation Instant Nonfat Dry Milk) was attached to the catheter and the solution injected retrograde from the tip of the catheter as NIRF was imaged using the system described above. FIG. 4 is a time-lapse composite as the renal pelvis filled with the ICG-in-milk enhanced fluorescent composition (“ICG-milk”) enhanced fluorescent composition. Fine detail of the anatomy can be seen beneath the overlying peritoneum, thus demonstrating both the successful penetration of excitation light and the capture of the resultant emitted fluorescence.

Fluorescence of an ICG-in-Enfamil Enhanced Fluorescent Composition.

As noted above, sterile infant formulas can be instilled for intraoperative detection of leaks. Enfamil is a sterile infant formula containing nonfat milk, lactose, and vegetable oils as major ingredients.

A vial of ICG containing 2.5 mg of ICG was dissolved into 10 ml of DMSO. This solution is intensely fluorescent and fluorescence can persist for at least six months. Aqueous solutions fade rapidly. The ICG in DMSO (“ICG/DMSO”) stock solution was diluted into Enfamil and fluorescence intensity measured. FIG. 5 shows that peak fluorescence the ICG-in-Enfamil enhanced fluorescent composition (“ICG-Enfamil”) was at 7.8 μg/ml similar to the peak seen in reconstituted dry non-fat milk. The DMSO concentration at the peak fluorescence was 0.3%. Thus enhancement of fluorescence intensity was similar to that observed with use of reconstituted nonfat dry milk.

Detection of Kidney Injury Using an ICG-in-Enfamil Enhanced Fluorescent Composition.

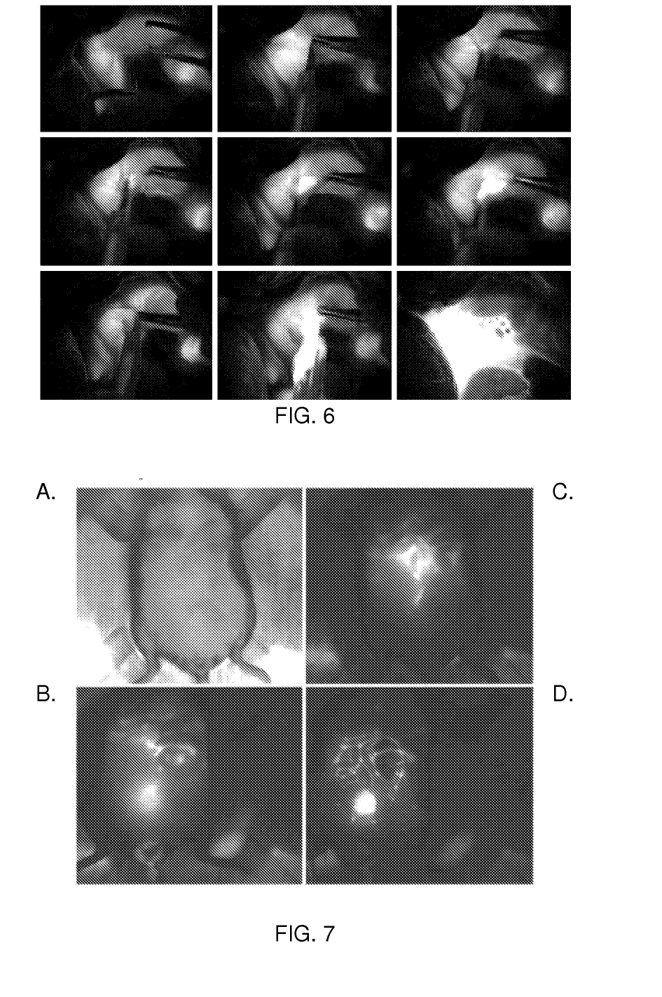

The utility of an ICG-in-Enfamil enhanced fluorescent composition (“ICG-Enfamil”) for visualization of ureteral and renal pelvis anatomy was evaluated in a live anesthetized pig. The ICG/DMSO stock solution was diluted into Enfamil to 7.5 μg/ml. The bladder of the pig was opened and a ureteral catheter inserted into the right ureter. The catheter was filled with the ICG-in-Enfamil enhanced fluorescent composition and the ureter and renal pelvis was clearly identified by NIRF imaging. A small incision was made in the renal pelvis and the leakage of the ICG-in-Enfamil enhanced fluorescent composition dramatically documented the kidney injury (FIG. 6).

Enhanced Fluorescence of ICG-in-Intralipid Enhanced Fluorescent Composition.

Intralipid is milk-like in its appearance and the major ingredient is 20% soy bean oil. Because Intralipid is approved for human intravenous use, a series of diluted solutions was prepared from the ICG/DMSO stock solution. Enhanced fluorescence was observed with a peak at ˜4 μg/ml.

Intraperitoneal Injection and Bladder Instillation of ICG-in-Intralipid Enhanced Fluorescent Composition.

An ICG-in-Intralipid enhanced fluorescent composition (“ICG-Intralipid”) was prepared from a 2.5 mg/ml ICG in DMSO stock solution by dilution into Intralipid to a final concentration of 5 μg/ml. A female mouse was euthanized by carbon dioxide inhalation and positioned under a NIRF imaging system.

FIG. 7 shows the reflected light image of the mouse after removal of abdominal hair. The ICG-in-Intralipid enhanced fluorescent composition (0.5 ml) was injected intraperitoneally (IP) and abdominal pooling of the enhanced fluorescent solution and outline of stomach and bowel were clearly visible through the skin. A catheter was then inserted into the urethra and 0.3 ml of ICG-Intralipid instilled into the bladder. A midline incision was then made in the skin and the ICG-in-Intralipid enhanced fluorescent composition was observed to be pooled around the intestines and retained in the bladder.

Intraperitoneal Injection and Excretion of ICG-in-Intralipid Enhanced Fluorescent Composition.

Female mice were anesthetized using isofluorane. Hair was removed from the abdomen and thorax using a depilatory and the mouse positioned in a NIRF imaging system (using Semrock ICG-A excitation and emission filters, and a Prosilica GC1380 camera equipped with a Navitar Zoom 7000 lens). An ICG-in-Intralipid enhanced fluorescent composition was injected IP (0.5 ml of 5 μg/ml ICG) under isoflurane anesthesia and NIRF images were made of the localization of the ICG-in-Intralipid enhanced fluorescent composition in the mouse.

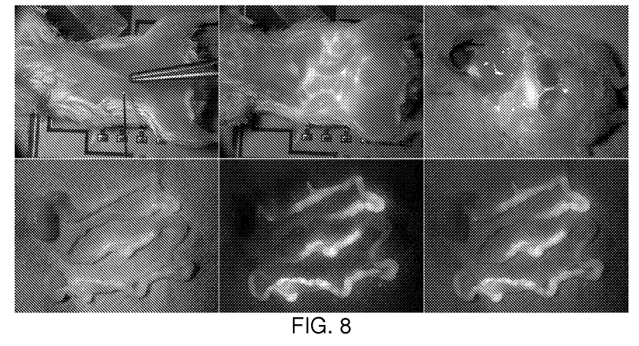

The resulting NIRF images are presented in FIG. 8. Following injection, the pooled ICG-in-Intralipid enhanced fluorescent composition clearly outlined the bowel (FIG. 8, middle top) and peristalsis was readily visualized through the skin. The mouse was allowed to recover from anesthesia and two hours later was re-anesthetized and the abdomen opened. The ICG-in-Intralipid enhanced fluorescent composition was still pooled in the abdomen. The gall bladder and contents of the intestine showed the ICG had been absorbed and eliminated in the bile. The bottom left panel of FIG. 8 is a reflected near-infrared light images of the excised bowel, the middle panel shows the intense fluorescence of the bowel contents and the right panel is a gray-scale version of a pseudocolored merge of the reflected and fluorescence images. It is clear that ICG from intraperitoneal ICG-Intralipid is eliminated by the expected hepatobiiary route.

Discussion

Near-infrared imaging and particularly fluorescence imaging expand the useful range of the electromagnetic spectrum beyond the visual range. ICG is attractive for use in biological applications of NIRF owing to its optical properties and established human safety profile. The results presented in this example demonstrate the utility of ICG in non-intravenous use for visualization of lumenal spaces.

In serum, the quantum efficiency of ICG fluorescence is high compared to that of aqueous solutions. Mixing ICG with milk, Enfamil, or Intralipid produces similar gains in quantum efficiency and our formulations contain components that have a long history of safe use.

Although fluorescent solutions of dye and milk were only made with concentrations of milk as high as 30%, there may be a benefit to making solutions of ICG and milk (e.g., Enfamil or powdered milk) or Intralipid having higher concentrations of milk or Intralipid, such as 40%, 50%, 60%, 70%, 80% or 90%. As indicated by FIG. 1, fluorescent solutions with concentrations of milk higher than 30% are likely to have higher fluorescent intensity. Fluorescent solutions with higher concentrations of ICG above 20 μg/mL can also be made, although the experimental data indicate that fluorescent intensity falls between an ICG concentration of about 5 and 10 μg/ml.

In experiments, the fluorescent intensities of 10%, 20% and 30% milk solutions were all higher at 5 μg/ml concentration of ICG than at 10 μg/ml concentration of ICG. At 5 μg/ml ICG and 10% milk concentration, the maximum fluorescent intensity was about 950000 (arbitrary units of integrated density IntDen), at 5 μg/ml ICG/20% milk, it was about 1000000 IntDen, while at 5 μg/ml ICG/30% milk concentration, the maximum fluorescent intensity was about 1150000 IntDen, or between about 1 and 2 orders of magnitude greater than the fluorescent intensity of a 1% milk/100 μg/mL ICG solution, a significant increase in fluorescent intensity.

Although powdered nonfat dry milk was used to make the milk solution solvent used above, non-powdered milk can also be used. Such milk can be sterilized and treated to make it appropriate for medical applications. Non-bovine milk, such as sheep or goat milk, can also be used to enhance the fluorescence of a dye such as ICG.

Intralipid, a branded emulsion of soy bean oil, egg phospholipids and glycerin, can be added to ICG instead of milk to provide a similar enhancement of fluorescence. When administered to mice by gavage, a mixture of Intralipid 20% and ICG was not absorbed from the GI track. Mixtures of ICG and other diluents such as fat emulsions and protein rich liquids can result in enhanced fluorescence and can be useful for the same purposes as the enhanced fluorescent compositions discussed above.

Visualization of the ureters with NIRF using the milk-ICG solution is a simple technique likely to facilitate identification of ureters during colorectal and gynecological surgeries. With the addition of powdered milk, the ICG fluorescence intensity was greatly enhanced. At low concentration (5 μg/mL ICG), it is sufficient to provide a sensitive signal to background ratio. Only a small volume (<10 mL) of the solution is needed to visualize the entire genital urinary tract.

This technique has several advantages. First, the ureter is directly visualized as opposed to identification via palpation with prophylactic stent insertion. Second, it allows immediate visualization of the ureters at any given time during the operation. If a ureteral injury is suspected during surgery, it allows surgeons to visualize the leakage and pinpoint an obstruction site immediately.

ICG displayed the highest fluorescent intensity at 5 μg/mL concentration. Florescent signal strengthens with increasing milk concentration. This example also demonstrates the successful visualization of ureters using NIRF with retrograde injections of 10% milk ICG solution on a human cadaver. The demonstration indicates the feasibility of using milk to enhance NIRF and as a tool to aid in preventing ureteral injuries during colorectal and gynecological surgeries.

ICG-milk solutions provide significantly enhanced fluorescence over ICG alone. Because both sterile milk and ICG are approved for use medically, ICG-milk solutions may be used in a number of medical applications where ICG alone or other fluorescent dyes are currently used, including angiography in ophthalmology (also diagnosis of corneal abrasions, corneal ulcers and herpetic corneal infections, or to evaluate the tear layer under rigid gas permeable contact lenses); non-invasive monitoring of liver or splanchnic perfusion; perfusion diagnostics of tissues and organs; navigation for sentinel lymph node biopsy with tumors; angiography to diagnose and categorize vascular disorders (in, for example, legs), retinal disease macular degeneration, diabetic retinopathy, inflammatory intraocular conditions, and intraocular tumors; during surgery for brain tumors; and diagnosis of rheumatic diseases.

Use of fluorescence enhanced solutions incorporating light scattering emulsions may result in enhanced depth of imaging through either enhanced collection or excitation wavelengths or return of emitted fluorescence.

The enhanced fluorescence of ICG-milk solutions also makes possible other applications, such as for nephrostomies, particularly in small mammals. In this application, a polymer tube is surgically inserted into the kidney, secured in place (such as with cyanoacrylate glue), and then the small mammal (e.g., rat) can be used for various types of laboratory experiments. The improved fluorescence enables better placement of the tube, by allowing visualization of the tubes position and evaluation of its connection to the renal pelvis.

Administration of infant feeding mixtures with small amounts of ICG may be used in neonatal intensive care settings for visualization of gastrointestinal function without the use of ionizing radiation, and may be useful for detection of abnormal anatomy or function. Transurethral or rectal administration may also find applications. Intravaginal, intrauterine, and fallopian administration will be of use in the management of fistula and/or other anatomical and/or functional defects in these structures, including the diagnosis and repair of rectovaginal and genitourinary fistulas (urethrovaginal, vesicovaginal, ureterovaginal, colovesical) and urethral diverticula. Administration by mouth may be useful for gut transport and continuity studies, especially in the neonatal intensive care unit. The safety of this route is likely as there is no enterohepatic reabsorption and mixtures administered by gavage do not result in the fluorescence of the bile ducts or gall bladder (murine studies and images). IP administration of Intralipid mix resulted in gall bladder and biliary tract fluorescence, so abdominal spills would result in predicted normal clearance patterns. As demonstrated in the mouse in this example, intraperitoneal administration provides ready visualization of position and movements of abdominal organs; this will be useful for guiding transabdominal procedures (FIG. 8).

Studies have demonstrated that intratracheal administration results in movement good back towards the head consistent with mucocilary transport. Thus this method may be useful as a test of airway clearance that does not rely on the use of radioactive materials or tissue harvest.

### 6.2 Example 2

### Enhanced Fluorescent Compositions

This example describes further characterization of the enhanced fluorescent compositions.

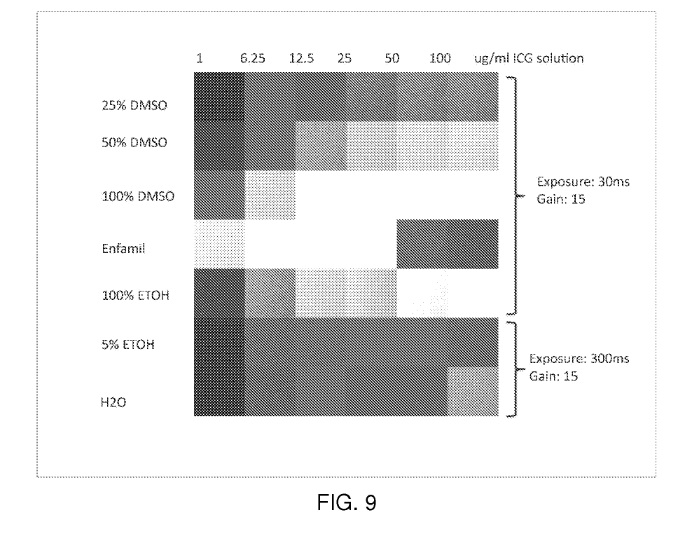

In the work summarized in FIG. 9, solutions of ICG were prepared in DMSO, water, or ethanol at 2.5 mg/ml and then diluted to the indicated concentrations across the top of the figure in the diluents listed down the left side. The DMSO and ethanol diluents were diluted with water. Two ml of each solution was transferred to a Petri dish and imaged as described herein in an NIRF imaging system. As shown in FIG. 9, at 25% DMSO, the intensity of fluorescence increased from 1 to 100 μg/ml, as indicated by this gray-scale version of the original color gradient. The same was observed at 50% DMSO. The imaging system was fully saturated at 12.5 μg/ml in 100% DMSO. Fluorescence in Enfamil was saturated between 6.25 and 25 μg/ml. Note, however, that it decreased, i.e., quenched, at 50 and 100 μg/ml. Intensity in 100% ethanol was lower than in DMSO but increased up to 100 μg/ml. Also imaged were the same ICG concentrations in 5% ethanol and water. To obtain signal the exposure time was increased from 30 ms to 300 ms to images these solutions. The range of fluorescence intensity in these various formulations exceeds the dynamic range of the imaging system. The use of ethanol and DMSO in pharmaceuticals is limited by the toxicity of these compounds. Therefore, while they do enhance ICG fluorescence, it is not possible to use them with dilution to very low concentrations. The fluorescence intensity of low concentrations of ICG in Enfamil, however, is quite favorable.

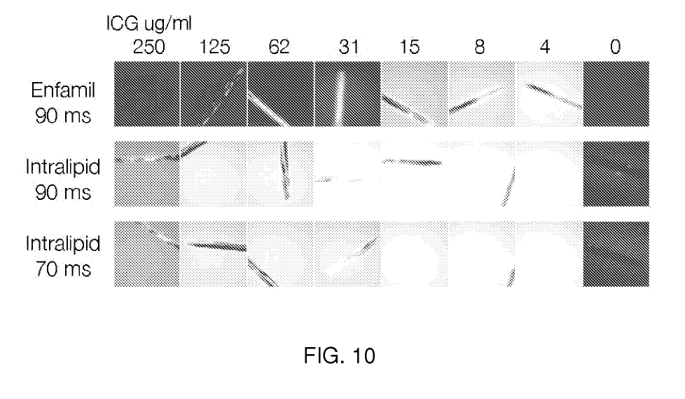

FIG. 10 shows images captured as described for FIG. 9, with the inclusion of a floating toothpick to provide an object with sharp edges to obtain optimal focus. ICG was dissolved in DMSO and then diluted into Enfamil or Intralipid. Concentrations decrease from right to left. In FIG. 10, the intensity of ICG fluorescence in Enfamil is not saturated and the peak around 8 μg/ml can be seen. Under these conditions, fluorescence saturated in Intralipid, but a peak at 8 μg/ml can be seen by reducing the exposure time from 90 to 70 ms.

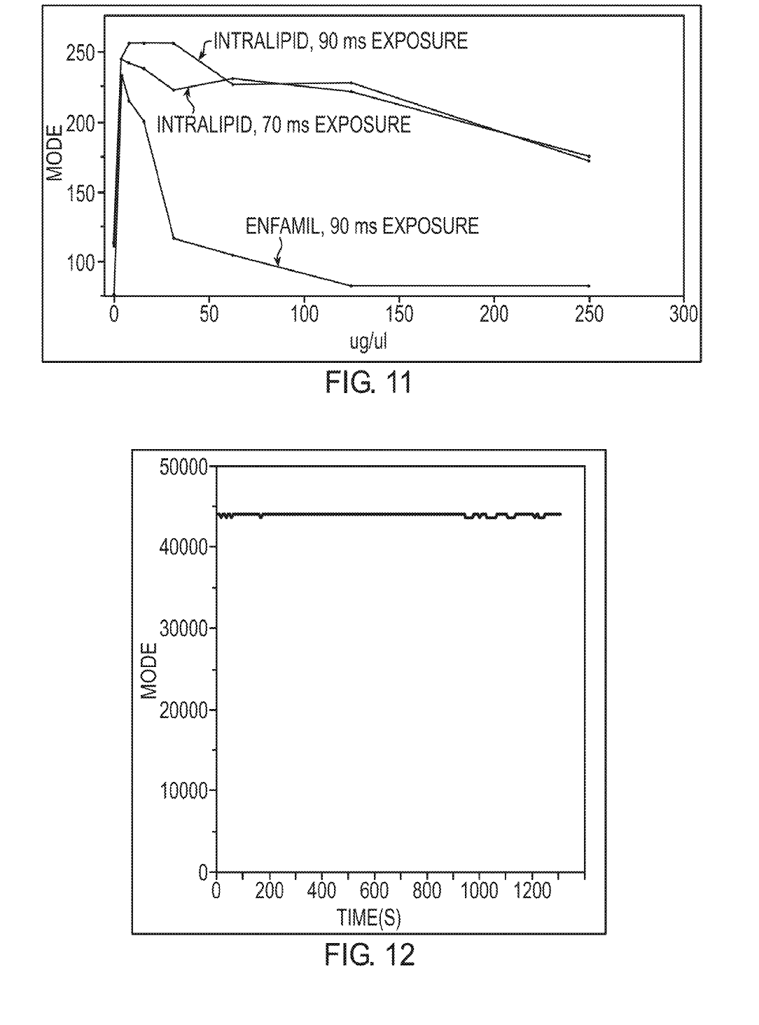

FIG. 11 is a graphical presentation of the fluorescence values (modes) in the image montages shown in FIG. 10.

With respect to FIG. 12, a solution of ICG in Enfamil (8 μg/ml) in a Petri dish was positioned in the imaging system. With constant illumination the fluorescence image was captured every 10 seconds for 22 minutes. In the graph shown in FIG. 12, only minor fluctuations are seen, demonstrating the stability of the fluorescence.

### 6.3 Example 3

### Visualization of Lumen During Surgery

This example describes a protocol for using an enhanced fluorescent composition comprising ICG for lumen (in this case the ureter) visualization during surgery in the vicinity of the lumen. This protocol can be used to visualize the lumen so that the surgeon does not injure the lumen accidently due to an inability to visualize it.

Retrograde Injection of ICG-Milk Enhanced Fluorescent Composition into the Ureter

The procedure is the same as a cystoscopy with ureteral catheterization and retrograde injection, which is well known in the art. Patient is covered with sterile drapes. A rigid or flexible cystoscope is passed per urethra. Ureteral orifices are identified and catheterized with an open ended catheter. It is usually only necessary to pass the catheter about 2 cm up the ureter but it can be passed up to the renal pelvis depending on area of interest. A guide wire can be used for difficult-to-catheterize orifices.

The catheter can be primed with air bubbles to remove the ICG-milk enhanced fluorescent composition.

The ICG-emulsion solution is then injected through the catheter using a syringe. When the procedure is concluded, the catheter is removed. Alternatively, the catheter can be left up the ureter with the distal tip outside the urethra so that it is positioned for a future injection.

The fluorescence of the ICG-emulsion enhanced fluorescent composition is then visualized with an NIRF imaging system, such as the Firefly, SPY, or Pinpoint instruments, or NIRF goggles.

### LITERATURE CITED

Al-Awadi K, Kehinde E O, Al-Hunayan A, Al-Khayat A. latrogenic ureteric injuries: incidence, aetiological factors and the effect of early management on subsequent outcome. Int Urol Nephrol. 2005; 37:235.
Augustin, A. J., Krieglstein, G. K.: Augenheilkunde, 2001, Springer-Verlag, ISBN 3-540-65947-1
Bothwell W N, Bleicher R J, Dent T L. Prophylactic ureteral catheterization in colon surgery. A five-year review. Dis Colon Rectum. 1994 April; 37(4):330-4.
Brandes S, Coburn M, Armenakas N, McAninch J. Diagnosis and management of ureteric injury: an evidence-based analysis. BJU Int. 2004; 94:277.
Crane L M, Themelis G, Arts H J, Buddingh K T, Brouwers A H, Ntziachristos V, van Dam G M, van der Zee A G. Intraoperative near-infrared fluorescence imaging forsentinel lymph node detection in vulvar cancer: first clinical results. Gynecol Oncol. 2011 February; 120(2):291-5. Epub 2010 November 6.
Duck F A: Properties of tissue including ultraviolet & infrared radiation, in Physical properties of tissue, London, Press A, Editor, 1990.
Hirche C, Dresel S, Krempien R, Hünerbein M: Sentinel node biopsy by indocyanine green retention fluorescence detection for inguinal lymph node staging of anal cancer: preliminary experience. Ann Surg Oncol. 2010 September; 17(9):2357-62. Epub 9. März 2010b.
Hirche C, Murawa D, Mohr Z, Kneif S, Hünerbein M. ICG fluorescence-guided sentinel node biopsy for axillary nodal staging in breast cancer. Breast Cancer Res Treat. 2010 June; 121(2):373-8. Epub 7. February 2010a.
Hutteman M, Mieog J S, van der Vorst J R, Liefers G J, Putter H, Löwik C W, Frangioni J V, van de Velde C J, Vahrmeijer A L. Randomized, double-blind comparison of indocyanine green with or without albumin premixing for near-infrared fluorescence imaging of sentinel lymph nodes in breast cancer patients. Breast Cancer Res Treat. 2011 May; 127(1):163-70.
Kelly H A. Operative Gynecology page 419. Second Edition, D. Appleton and Company,
1906.
Kim S, Lim Y T, Soltesz E G, De Grand A M, Lee J, Nakayama A, et al. Near-infrared fluorescent type II quantum dots for sentinel lymph node mapping. Nat Biotechnol. 2004; 22:93.
Kovac S R. Advances in Reconstructive Vaginal Surgery, 2006.
Kuno K, Menzin A, Kauder H H, Sison C, Gal D. Prophylactic ureteral catheterization in gynecologic surgery. Urology. 1998 December; 52(6):1004-8.
McLoughlin, M G, Williams, G M. Infusion of Sterile Milk to Document Watertight Urothelial Closure. Urology. 1976 May; 7(5):525.
Rao J, Dragulescu-Andrasi A, Yao H. Fluorescence imaging in vivo: recent advances. Curr Opin Biotechnol. 2007 February; 18(1):17-25. Epub 2007 January 17
Tanaka, E., Ohnishi, O., Laurence, R. G., Choi, H. S., Humblet, V., Frangioni, J. V. Real-Time Intraoperative Ureteral Guidance Using Invisible Near-Infrared Fluorescence J. Urol. 2007 November; 178(5): 2197-2202.
Tobis S, Knopf J, Silvers C, Yao J, Rashid H, Wu G, Golijanin D. Near infrared fluorescence imaging with robotic assisted laparoscopic partial nephrectomy: initial clinical experience for renal cortical tumors. J Urol. 2011 July; 186(1):47-52.
Wood E C, Maher P, Pelosi M A. Routine use of ureteric catheters at laparoscopic hysterectomy may cause unnecessary complications. J Am Assoc Gynecol Laparosc. 1996; 3:393.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. An enhanced fluorescent composition comprising:
a fluorescent dye; and
fluorescence-enhancing diluent (“FED”) comprising a solution, emulsion or suspension fat, oil, or protein in liquid.

2. The fluorescent composition of claim 1 wherein the liquid is water, saline solution, ethanol or DMSO.

3. The fluorescent composition of claim 1 wherein the fluorescence-enhancing diluent comprises powdered, freeze-dried, dehydrated, evaporated, concentrated or condensed fat, oil, or protein in liquid.

4. The fluorescent composition of claim 1 wherein the fluorescent dye is a near-infrared fluorescent dye.

5. The fluorescent composition of claim 1 wherein the fluorescent dye is indocyanine green (ICG).

6. The fluorescent composition of claim 1 wherein the fluorescence-enhancing diluent comprises a fat emulsion comprising one of milk, infant formula, intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

7. The fluorescent composition of claim 6 wherein the milk is cow, sheep or goat milk.

8. The fluorescent composition of claim 1 wherein the concentration of fluorescence-enhancing diluent in the fluorescent composition is 0.1% to 90% v/v.

9. The fluorescent composition of claim 1 wherein the concentration of fluorescence-enhancing diluent is between 5% and 15% (±5%) v/v.

10. The fluorescent composition of claim 5 wherein the concentration of ICG in the enhanced fluorescent composition in μg/mL is between 5% and 40% (±3%).

11. The fluorescent composition of claim 5 wherein the ICG concentration in μg/mL is between 5% and 15% (±3%).

12. The fluorescent composition of claim 1 wherein the fluorescent intensity of the enhanced fluorescent composition is at least 2, 3, 4, 5, 6, 7, 8, 10, 15, or 20 times that of a fluorescent solution containing the same amount of fluorescent dye but no fluorescence-enhancing diluent.

13. The fluorescent solution of claim 1 that is micro-encapsulated.

14. A method for enhancing fluorescent intensity of a fluorescent dye comprising the step of mixing the fluorescent dye with fluorescence-enhancing diluent, thereby producing an enhanced fluorescent composition.

15. The method of claim 14 comprising the step of mixing the fluorescent dye with a liquid.

16. The method of claim 15 wherein the liquid is water, saline solution, ethanol or DMSO.

17. The method of claim 14 wherein the fluorescent dye is a near-infrared fluorescent dye.

18. The method of claim 14 wherein the fluorescent dye is indocyanine green (ICG).

19. The method of claim 14 wherein the fluorescence-enhancing diluent is milk, infant formula, intravenous fat emulsions, soy bean oil, egg phospholipids, Intralipid, Liposyn, Nutralipid, Soyacal, Travamulsion, SMOFlipid, Clinoleic, Lipovenoes and/or combinations thereof.

20. The method of claim 19 wherein the milk is cow, sheep or goat milk.

21. The method of claim 14 wherein the fluorescence-enhancing diluent is a solution of powdered, freeze-dried, dehydrated, evaporated, concentrated or condensed fat emulsion in liquid.

22. The method of claim 14 wherein the concentration of fluorescence-enhancing diluent in the fluorescent composition is 0.1% to 90% v/v.

23. The method of claim 14 wherein the concentration of fluorescence-enhancing diluent is between 5% and 15% (±5%) v/v.

24. The method of claim 14 wherein the fluorescent dye is ICG and the concentration of ICG in μg/mL is between 5% and 40% (±3%).

25. The method of claim 24 wherein the ICG concentration in μg/mL is between 5% and 15% (±3%).

26. The method of claim 14 wherein the fluorescent intensity of the enhanced fluorescent composition is at least 2, 3, 4, 5, 6, 7, 8, 10, 15, or 20 times that of a fluorescent solution containing the same amount of fluorescent dye but no fluorescence-enhancing diluent.

27. The method of claim 14 comprising the step of micro-encapsulating the enhanced fluorescent composition.

28. The fluorescent composition of claim 1 wherein the fluorescent dye is a carbocyanine dye.

29. The fluorescent composition of claim 1 wherein the fluorescence-enhancing diluent is a suspension of nonfat powdered milk in a liquid.
